(19) United States
(12) Patent Application Publication
(10) Pub. No.: US 2009/0317443 A1
(43) Pub. Date: Dec. 24, 2009

(54) COATED IMPLANT

(75) Inventors: Sean Willis, Surrey (GB); Vincent James O'Byrne, Surrey (GB); Samantha Ryan, Surrey (GB)

Correspondence Address: SUGHRUE MION, PLLC, 2100 PENNSYLVANIA AVENUE, N.W., SUITE 800, WASHINGTON, DC, 20037, US

(73) Assignee: BIOCOMPATIBLES UK LIMITED Chapman House, Surrey (GB)

(21) Appl. No.: 12/373,334

(22) PCT Filed: Jul. 16, 2007
(86) PCT No.: PCT/EP07/57333

§ 371 Date: Feb. 9, 2009

(30) Foreign Application Priority Data
Jul. 14, 2006 (EP) 06253705.5

(51) Int. Cl. A61K 9/00; B05D 3/00; B05D 1/36; A61P 43/00; A61F 2/82

(52) U.S. Cl. 424/423; 427/2.24; 427/2.25; 623/1.46; 623/1.42

(57) ABSTRACT

A method forming a coated implant is described. The implant comprises a surface which is first coated with a primer and subsequently with a biocompatible polymer capable of forming a covalent bond to the primer. The polymer coating is then crosslinked. The invention also relates to implants, in particular, stents, coated with such a coating.

The present invention relates to a coated implant for implantation into an animal, for instance a human, and to methods for producing the same.

A leading cause of mortality within the developed world is cardiovascular disease. Patients having such disease usually have narrowing in one or more coronary arteries. One treatment is coronary stenting, which involves the placement of a stent at the site of acute artery closure. This type of procedure has proved effective in restoring vessel patency and decreasing myocardial ischaemia. However, exposure of stents—especially those made of metals—to flowing blood can result in thrombus formation, platelet activation and acute thrombotic occlusion of the stent.

Our research has focussed on providing suitable coatings for commonly used stents and other implants. It is important that such coatings are mechanically stable and bio-inert.

Non-thrombogenic and anti-thrombogenic coatings for stents have been developed. Stents have been coated with polymers having pendant zwitterionic groups, specifically phosphorylcholine (PC) groups, generally described in WO-A-93/01221. A particularly successful embodiment of those polymers suitable for use on stents has been described in WO-A-98/30615. The polymers coated onto the stent have pendant crosslinkable groups which are subsequently crosslinked by exposure to suitable conditions, generally heat and/or moisture. Specifically a trialkoxysilylalkyl group reacts with pendant groups of the same type and/or with hydroxyalkyl groups to generate intermolecular crosslinks.

One very important feature of polymer coatings on medical devices is their ability to firmly bond to the surface of the implant and remain adhered even when subjected to either mechanical or biological challenge in either the in vitro or in vivo environment. There have been suggestions as to how this adhesion can be improved further.

One solution is to use a primer between the implant surface and the polymer coating. More specifically, a stent surface may be coated with a primer in order to improve adhesion. For example, in U.S. Pat. No. 5,380,299, a stent is provided with a coating of a thrombolytic compound and optionally an outer layer of an anti-thrombotic compound. The stent may be precoated with a “primer” such as a cellulose ester or nitrate. U.S. Pat. No. 6,723,373 describes a process for coating stents with a silicone polymer whereby in some of the exemplified embodiments, the stent is precoated with a silicone adhesion primer, specifically SP1 from Nusil.

The present applicant has focussed on trying to improve the adhesion of the coating onto the stent by investigating both the nature of the primer and the polymer which forms the coating. In doing so, a new combination of polymer and primer and implant has been developed which provides beneficial results.

In US 20030143335 biomedical articles such as contact lenses are coated with a polymeric tie layer having reactive sites and then a top coat having sites which react with the tie layer reactive sites. The tie layer is a polyelectrolyte and adheres to the surface electrostatically. The top coat may react by various means with the tie layer although the only specific example reacts electrostatically. The present invention provides a method of forming a coated implant, whose surface comprising the following steps:
(i) coating the surface of the implant with a primer to form a primer layer after cleaning and/or plasma treating the surface;
(ii) coating the primer layer with a biocompatible polymer having a functional pendant group capable of forming a covalent bond with the primer;
(iii) forming a covalent bond between the primer and the biocompatible polymer; and
(iv) covalently crosslinking the polymer coating.

In the method of the present invention, the first essential step is to coat the surface(s) of the implant with a primer to form a primer layer. The primer may be coated on either the exterior or interior surfaces of the implant depending on to which walls of the implant the polymer coating will ultimately be applied. Examples of suitable primers for use in the method of the present invention include primer compounds including a monoalkoxysilyl group, a dialkoxysilyl group, a trialkoxysilyl group, a triacyloxysilyl group, and/or a chloro silyl group. Mixtures of compounds may be used. The composition coated onto the surface generally contains a solvent, preferably a volatile solvent which is removed by evaporation. A primer is often cured after coating to at least partially cross-link the primer compounds. Such primers can be obtained commercially from, for instance, Aldrich Chemicals and Nusil Technology Corp.

Without wishing to be bound by theory, it is believed that such primer compounds react by forming a silanol substituted primer intermediate which then reacts with the pendant functional group of the biocompatible polymer to form covalent bonds. During application of the primer and the polymer, and during subsequent processing, conditions are controlled so as to allow for covalent bond formation between the primer and the polymer.

In a preferred embodiment of the present invention, either or both of the primer compound and the biocompatible polymer include a pendant group of general formula (II)

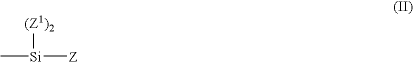

wherein Z is —OR³⁰ or Hal
Z¹ is —OR³⁰, Hal or C₁₋₁₂ alkyl

wherein R³⁰ is C₁₋₁₂ alkyl or acyl and Hal is a halogen atom. R³⁰ may be substituted by C₁₋₄ alkoxy or hydroxy.

In a particularly preferred embodiment of the invention either or both of the primer compound and the biocompatible polymer include a pendant group of general formula (IIA)

—Si(OR⁵)₃ (IIA)

wherein R⁵ is C₁₋₁₂ alkyl or C₂₋₁₂ acyl

The primer may comprise a mixture of compounds such as silicate, a titanate or zirconate, and a silane having a group of general formula II or IIA. In one embodiment of the present invention, where the primer includes a compound having a group of formula (II), the silane primer comprises a mixture of tetra-n-propyl silicate, tetrabutyltitanate and tetra (2-methoxyethoxy)silane (the primer compound having a pendant group of general formula II) along with a solvent. An example of such a commercially available primer is SP120 which is available from Nusil Technology Corp. USA. Without wishing to be bound by theory, it is thought that this primer works particularly well because the titanate in the mixture acts as a catalyst to the cross-linking reaction of the polymer.

A further example of a particularly preferred primer compound having a group of general formula II is bis[3-(trimethoxysilyl)propyl]amine (BTMSPA).

The primer is applied to the wall of the implant by conventional liquid coating techniques such as, for example, dip coating, spray coating and spin coating. Preferably, the primer is applied so as to give a coating thickness of up to 100 nm, for instance in the range from 5 to 30 nm, more preferably, 10 to 20 nm, most preferably 12 to 16 nm. The primer layer may be coated with the biocompatible polymer in step (ii) either before or after it has been dried.

Optionally, indeed preferably, the process includes a preliminary cleaning step, in which the implant surface is cleaned before the coating with primer. This may help improve the adhesion of the overall coating. Suitable cleaning steps involve the use of solvents and/or surfactants. The cleaned surface is usually rinsed and dried before primer treatment. The surface may additionally or alternatively be plasma treated, for instance using an oxygen plasma. In some instances a plasma treatment step can be used to improve adhesion further.

The second step in the method according to the present invention, step (ii), is to coat the primer layer with a biocompatible polymer. As detailed above, the primer and biocompatible polymer must be selected such that a covalent bond is formed between the primer layer and biocompatible polymer layer. The formation of the covalent bond may take place at the same time as the crosslinking step. It is thought that the formation of a covalent bond leads to an improved adhesion bond to the surface of the implant thus minimising the likelihood of later delamination.

The biocompatible polymer may be biostable, biodegradable or bioerodable. Once cross-linked, the polymer is preferably water-insoluble and is water-swellable. The polymer may, for example, be a silicone hydrogel; a polyurethane; a polysaccharide, such as an alginate; a polyether such as polyethylene glycol; a polyamide or polyester, such as a hydroxybutyric acid polymer or copolymer; or a poly(lactide) or poly(glycolide). The polymer is cross-linkable, either by virtue of having pendant groups capable of forming inter- or intra-molecular crosslinking, or by having functional groups which may be reacted with extrinsic di- or higher-functional cross-linking agents. Preferably the polymer is formed from ethylenically unsaturated monomers, more preferably including a zwitteronic monomer, and a reactive monomer having general formula (I). The reactive monomer leads to the polymer being crosslinkable as well as being reactive with the primer.

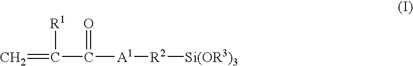

In a preferred embodiment, the biocompatible polymer is obtained by polymerising monomers, including at least one monomer unit having a pendant group of the formula (II). Furthermore, in a preferred embodiment, the biocompatible polymer is obtained by copolymerising ethylenically unsaturated monomers including at least one monomer having the general formula (I)

in which R¹ is hydrogen or C₁₋₄ alkyl;

A¹ is —O— or —NR⁴ wherein R⁴ is hydrogen or C₁₋₄ alkyl;

R² is C₁₋₂₄ straight or branched alkylene, alkylene oxaalkylene or alkylene oligooxaalkylene in which the or each alkylene group has 1 to 6 carbon atoms; and

each R³ is independently selected from C₁₋₆ alkyl groups.

In formula (I), R¹ is preferably selected from hydrogen and methyl, most preferably R¹ is methyl. A¹ is preferably —O—. R² is preferably C₂₋₆ alkylene, more preferably C₂₋₄ alkylene. R³ is selected from C₁₋₆-alkyl groups, preferably C₁₋₂ alkyl groups.

This monomer provides the functional pendant groups capable of forming a covalent bond with the primer and, further, with the cross-linkable groups, whereby cross-linking of the polymer coating may be performed. Activation of the functional group —Si(OR³)₃ by heat and/or moisture, for example results in inter- and intra-molecular cross-links being formed between polymer chains. This helps to produce a cross-linked polymer coating which is sufficiently robust and has a reduced tendency to flow under manufacturing and processing conditions at raised temperatures. The silyl groups of formula (I) interact with the primer layer, particularly where the primer is a silane primer, including groups of formula (II) to enhance adhesion and minimise delamination from the implant surface in use.

Preferably the ethylenically unsaturated monomer from which the biocompatible polymer is formed includes zwitterionic monomer having the general formula (III)

YBX (III)

wherein

B is a straight or branched alkylene (alkanediyl), alkyleneoxaalkylene or alkylene oligo-oxaalkylene chain optionally containing one or more fluorine atoms up to and including perfluorinated chains or, if X or Y contains a terminal carbon atom bonded to B, a valence bond;

X is a zwitterionic group; and

Y is an ethylenically unsaturated polymerisable group selected from

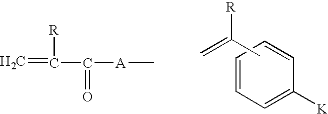

CH₂═C(R)CH₂O—, CH₂═C(R)CH₂OC(O)—, CH₂═C(R)OC(O)—, CH₂═C(R)—O—, CH₂═C(R)CH₂OC(O)N(R⁶)—, R⁷OOCCR═CRC(O)O—, RCH═CHC(O)O—, RCH═C(COOR⁷)CH₂C(O)O—,

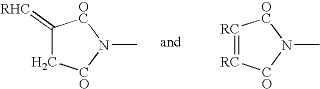

wherein:

R is hydrogen or a C₁-C₄ alkyl group;

R⁶ is hydrogen or a C₁-C₄ alkyl group or R⁶ is —B—X where B and X are as defined above; and

R⁷ is hydrogen or a C₁₋₄ alkyl group;

A is —O— or —NR⁶—;

K is a group —(CH₂)ₚOC(O)—, —(CH₂)ₚC(O)O—,

—(CH₂)ₚOC(O)O—, —(CH₂)ₚNR⁸—, —(CH₂)ₚNR⁸C(O)—,
—(CH₂)ₚC(O)NR⁸—, —(CH₂)ₚNR⁸C(O)O—, —(CH₂)ₚOC(O)NR⁸—, —(CH₂)ₚNR⁸C(O)NR⁸— (in which the groups R⁸ are the same or different), —(CH₂)ₚO—, —(CH₂)ₚSO₃—, or, optionally in combination with B, a valence bond

p is from 1 to 12; and

R⁸ is hydrogen or a C₁-C₄ alkyl group.

In group X, the atom bearing the cationic charge and the atom bearing the anionic charge are generally separated by 2 to 12 atoms, preferably 2 to 8 atoms, more preferably 3 to 6 atoms, generally including at least 2 carbon atoms.

Preferably the cationic group in zwitterionic group X is an amine group, preferably a tertiary amine or, more preferably, a quaternary ammonium group. The anionic group in X may be a carboxylate, sulphate, sulphonate, phosphonate, or, more preferably, phosphate group. Preferably the zwitterionic group has a single monovalently charged anionic moiety and a single monovalently charged cationic moiety. A phosphate group is preferably in the form of a diester.

Preferably, in a pendant group X, the anion is closer to the polymer backbone than the cation.

Alternatively group X may be a betaine group (ie in which the cation is closer to the backbone), for instance a sulpho-, carboxy- or phospho-betaine. A betaine group should have no overall charge and is preferably therefore a carboxy- or sulpho-betaine. If it is a phosphobetaine the phosphate terminal group must be a diester, i.e., be esterified with an alcohol. Such groups may be represented by the general formula (IV)

—X¹—R⁹—N⁺(R¹⁰)₂—R¹¹-Q (IV)

in which X¹ is a valence bond, —O—, —S— or —NH—, preferably —O—;

V is a carboxylate, sulphonate or phosphate (diester-monovalently charged) anion;

R⁹ is a valence bond (together with X¹) or alkylene —C(O)alkylene- or —C(O)NHalkylene preferably alkylene and preferably containing from 1 to 6 carbon atoms in the alkylene chain;

the groups R¹⁰ are the same or different and each is hydrogen or alkyl of 1 to 4 carbon atoms or the groups R¹⁰ together with the nitrogen to which they are attached form a heterocyclic ring of 5 to 7 atoms; and

R¹¹ is alkylene of 1 to 20, preferably 1 to 10, more preferably 1 to 6 carbon atoms.

One preferred sulphobetaine monomer has the formula (V)

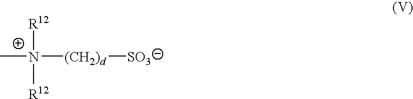

where the groups R¹² are the same or different and each is hydrogen or C₁₋₄ alkyl and d is from 2 to 4.

Preferably the groups R¹² are the same. It is also preferable that at least one of the groups R¹² is methyl, and more preferable that the groups R¹² are both methyl.

Preferably d is 2 or 3, more preferably 3.

Alternatively the group X may be an amino acid moiety in which the alpha carbon atom (to which an amine group and the carboxylic acid group are attached) is joined through a linker group to the backbone of the polymer A. Such groups may be represented by the general formula (VI)

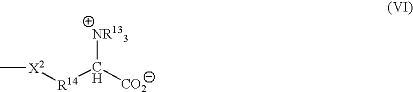

in which X² is a valence bond, —O—, —S— or —NH—, preferably —O—,

R¹³ is a valence bond (optionally together with X²) or alkylene, —C(O)alkylene- or —C(O)NHalkylene, preferably alkylene and preferably containing from 1 to 6 carbon atoms; and

the groups R¹³ are the same or different and each is hydrogen or alkyl of 1 to 4 carbon atoms, preferably methyl, or two of the groups R¹³, together with the nitrogen to which they are attached, form a heterocyclic ring of from 5 to 7 atoms, or the three group R¹³ together with the nitrogen atom to which they are attached form a fused ring structure containing from 5 to 7 atoms in each ring.

X is preferably of formula (VII)

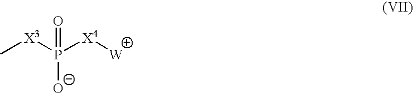

in which the moieties X³ and X⁴, which are the same or different, are —O—, —S—, —NH— or a valence bond, preferably —O—, and W⁺ is a group comprising an ammonium, phosphonium or sulphonium cationic group and a group linking the anionic and cationic moieties which is preferably a C₁₋₁₂-alkanediyl group.

Preferably W contains as cationic group an ammonium group, more preferably a quaternary ammonium group.

The group W⁺ may for example be a group of formula —W¹—N⁺R¹⁵₃—W¹—P⁺R¹⁶₃, —W¹—S⁺R¹⁶₂ or —W¹-Het⁺ in which:

W¹ is alkanediyl of 1 or more, preferably 2-6 carbon atoms optionally containing one or more ethylenically unsaturated double or triple bonds, disubstituted-aryl, alkylene aryl, aryl alkylene, or alkylene aryl alkylene, disubstituted cycloalkyl, alkylene cycloalkyl, cycloalkyl alkylene or alkylene cycloalkyl alkylene, which group W¹ optionally contains one or more fluorine substituents and/or one or more functional groups; and

either the groups R¹⁵ are the same or different and each is hydrogen or alkyl of 1 to 4 carbon atoms, preferably methyl, or aryl, such as phenyl or two of the groups R¹⁵ together with the nitrogen atom to which they are attached form a heterocyclic ring containing from 5 to 7 atoms or the three groups R¹⁵ together with the nitrogen atom to which they are attached form a fused ring structure containing from 5 to 7 atoms in each ring, and optionally one or more of the groups R¹⁵ is substituted by a hydrophilic functional group, and

the groups R¹⁶ are the same or different and each is R¹⁵ or a group OR¹⁵, where R¹⁵ is as defined above; or

Het is an aromatic nitrogen-, phosphorus- or sulphur-, preferably nitrogen-, containing ring, for example pyridine.

Preferably W¹ is a straight-chain alkanediyl group, most preferably ethane-1,2-diyl.

Preferred groups X of the formula (VII) are groups of formula (VIII):

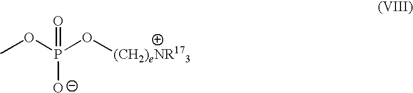

where the groups R¹⁷ are the same or different and each is hydrogen or C₁₋₄ alkyl, and e is from 1 to 4.

Preferably the groups R¹⁷ are the same. It is also preferable that at least one of the groups R¹⁷ is methyl, and more preferable that the groups R¹⁷ are all methyl.

Preferably e is 2 or 3, more preferably 2.

Alternatively the ammonium phosphate ester group VIII may be replaced by a glycerol derivative of the formula VB, VC or VD defined in our earlier publication no WO-A-93/01221.

Preferably the ethylenically unsaturated group Y is an acrylic type group, of the formula H₂C═C(R)C(O)-A. Preferably R is H or CH₃. Preferably A and A¹ are the same and are most preferably —O—. B is preferably straight chain C₂₋₆-alkanediyl.

Preferably the ethylenically unsaturated comonomers comprise diluent comonomers which may be used to give the polymer desired physical and mechanical properties. Particular examples of diluent comonomers include alkyl(alk)acrylate preferably containing 1 to 24 carbon atoms in the alkyl group of the ester moiety, such as methyl (alk)acrylate or dodecyl methacrylate; a dialkylamino alkyl(alk)acrylate, preferably containing 1 to 4 carbon atoms in each alkyl moiety of the amine and 1 to 4 carbon atoms in the alkylene chain, e.g. 2-(dimethylamino)ethyl (alk)acrylate; an alkyl (alk)acrylamide preferably containing 1 to 4 carbon atoms in the alkyl group of the amide moiety; a hydroxyalkyl (alk)acrylate preferably containing from 1 to 4 carbon atoms in the hydroxyalkyl moiety, e.g. a 2-hydroxyethyl (alk)acrylate glycerylmonomethacrylate or polyethyleneglycol monomethacrylate; or a vinyl monomer such as an N-vinyl lactam, preferably containing from 5 to 7 atoms in the lactam ring, for instance vinyl pyrrolidone; styrene or a styrene derivative which for example is substituted on the phenyl ring by one or more alkyl groups containing from 1 to 6, preferably 1 to 4, carbon atoms, and/or by one or more halogen, such as fluorine atoms, e.g. (pentafluorophenyl)styrene.

Other suitable diluent comonomers include polyhydroxyl, for example sugar, (alk)acrylates and (alk)acrylamides in which the alkyl group contains from 1 to 4 carbon atoms, e.g. sugar acrylates, methacrylates, ethacrylates, acrylamides, methacrylamides and ethacrylamides. Suitable sugars include glucose and sorbitol. Diluent comonomers include methacryloyl glucose and sorbitol methacrylate.

Further diluents which may be mentioned specifically include polymerisable alkenes, preferably of 2-4 carbon atoms, eg. ethylene; dienes such as butadiene; ethylenically unsaturated dibasic acid anhydrides such as maleic anhydride; and cyano-substituted alkenes, such as acrylonitrile.

Particularly preferred diluent monomers are nonionic monomers, most preferably alkyl(alk)acrylates or hydroxyalkyl(alk)acrylates.

It is particularly desirable to include hydroxyalkyl(alk)acrylates in combination with reactive comonomers which contain reactive silyl moieties including one or more halogen or alkoxy substituent. The hydroxyalkyl group containing monomer may be considered a reactive monomer although it also acts as a diluent. Such reactive silyl groups are reactive with hydroxy groups to provide crosslinking of the polymer after coating, for instance.

A particularly preferred biocompatible polymer for use in step (ii) is a crosslinkable polymer formed by free radical polymerisation of ethylenically unsaturated monomers including

i) a zwitteronic monomer of formula (III) wherein X is a group of formula (VII), preferably (VIII),

ii) styrene or a substituted styrene in an amount in the range from 5 to 40 wt %,

iii) 10 to 89 wt % of a monomer (a) or mixture of monomers (a, b etc) whose homopolymers having glass transition temperatures Tga etc together have a calculated Tg calculated using the formula

$$\frac{1}{Tg} = \frac{Wa}{Tga} + \frac{Wb}{Tgb} + …$$

which is lower than the Tg of a homopolymer formed from monomer (i) and lower that the Tg of a homopolymer formed by polymerizing monomer (ii)

wherein Tg a and Tbg=Tg of a homopolymer of a and b respectively (^{O}K), Wa and Wb=weight fraction of components a and b respectively in the mixture; and

iv) 0.1 to 10 wt % of a monomer having a crosslinkable group preferably a monomer of general formula I.

Preferably the monomer (a) or each of the groups of monomers (a, b etc) is selected from the monomers mentioned above as diluent monomers, for instance C₁₋₂₄ alkyl(alk)acrylates and -(alk)acrylamides and analogues having hydroxyl or (oligo) alkoxy substituents on the C₁₋₂₄ alkyl groups. More preferably at least one of the monomer, or each of the monomers is a C₄₋₁₂ alkyl(meth)acrylate, or hydroxy substituted C₄₋₁₂ alkyl(meth)acrylate. Preferably a (meth)acrylate or a (meth)acrylamide is an acrylate or acrylamide. This monomer (mixture) is referred to below as a low Tg monomer. Examples of suitable monomers are ethyl acrylate, ethyl methacrylate, methylmethacrylate, 2-hydroxy ethylmethacrylate, 2 ethyl hexyl acrylate, hydroxypropyl methacrylate lauryl methacrylate and PEG(meth)acrylates. Examples of Tgs of homopolymers are styrene 100° C., methylmethacrylate 105° C., butylacrylate −56° C., ethyl acrylate −22° C., 2-ethylhexylacrylate −70° C. Other Tgs are disclosed in Brandrup et al (eds) Polymer Handbook 4th Ed. (2003), John Wiley & Sons.

An example of a suitable biocompatible polymer is one obtained by copolymerising a mixture of 2-methacryloyloxyethyl-2′-trimethylammonium ethyl phosphate inner salt, styrene, methyl methacrylate, butyl acrylate, hydroxybutyl acrylate and trimethoxysilyl propyl methacrylate.

The biocompatible polymer may be applied to the surface of the implant in any of a number of ways. In one embodiment the implant is coated by dipping the surface into a solution containing the polymer. In an alternative embodiment of the present invention, the polymer may be applied by a spray process. The polymer solution which is sprayed on to the implant may further include one or more pharmaceutical actives. In this way, the polymer coating on the implant will also include pharmaceutical active(s). Other ways of loading a pharmaceutical active may be used, such as by dipping a polymer coated implant in a solution or dispersion of active.

Suitable liquid vehicles for coating compositions are solvents for the polymer, such as esters, alcohols, ethers, glycols or ketones, especially alcohols, such as C₂₋₆ alkanols, especially n- or i-propanol and ethanol, as well as mixtures, including mixtures with water or glycols. The method described in WO01/01957 which leads to thicker coatings on an external surface may be utilised.

Following step (ii), the polymer coating is cross-linked to form a polymer matrix. Cross-linking may be achieved by any known technique. Examples include the application of heat and/or moisture, ethylene oxide treatment, UV irradiation, gamma sterilisation, electron beam irradiation, and autoclaving. Cross-linking may be carried out before or after drug loading.

The or each pharmaceutical active is a compound which is required to be delivered to the location at which this implant is implanted. The polymer coating ensures that a controlled release of active is possible. This release may in part be controlled by the crosslinking to form a crosslinked polymer matrix.

Examples of suitable pharmaceutical actives include antibiotics, antiangiogenic compounds, anti-inflammatories, such as steroids or NSAIDS, e.g. COX inhibitors, glucocorticoids and corticosteroids, anti-platelet drugs, anti-coagulants, lipid reguating drugs, such as statins, cytotoxic drugs, such as antimetabolites, vinca alkaloids, other anti neoplastics, matrixmetallo proteinase inhibitors cyto toxic antibiotics, specific examples include rapamycin and analogues thereof such as RAD001, tacrolimus, everolimus, Biolimus A9 and zotarolimus; tyrphostin; angiopeptin; carmustine; flavopiridol; gemcitabine and salts, tecans, such as camptothecin, topotecan and irinotecan, lomustine, methotrexate, mitomycin, taxanes, such as paclitaxel and docetaxel; actinomycin D, vincristine, vinblastine, streptozotocin, capecitabine, vinorelbine, doxorubicin and other anthracyclines, dexamethasone and derivatives thereof, in particular hydrophobic derivatives and dexamethasone phosphate, mometasone, triamcinolone, clobestasol, tetradecylselenoacetic acid, tetradecylthioacetic acid, ethylisopropylamiloride, antithrombin, aggrastat, aspirin, cilostazol, clexane, clopidogrel, dipyridamole, persantine, integrillin (eptifibatide), abciximabs, trapidil (rocornal), matrixmetallo proteinase, such as batimastat, marimastat; growth factors such as VEGF; gene therapy agents; statins such as avostatin, cerivastatin, flavastatin, lovastatin, rosuvastatin, simvastatin and sandostatin, carvedilol, estradiol and methoxyestradiol, L-arginine, nitric oxide donors, probucol, quinaprilat, thioctacid, telmisartan, zoledronate, and mixtures thereof. Also, agents such as antibodies and peptide sequences which encourage a natural healing process can be used e.g. growth factors.

A stent is one example of an implant falling within the scope of the present invention. The term “stent” as used hereinafter is used to describe a stent as commonly understood in the present field. The stent is for permanent or temporary implantation into an animal, preferably a human body. Such stents comprise a generally tubular body having an interior wall and an exterior wall.

The polymer coating is preferably applied to at least the exterior surface and, where the implant is a hollow structure such as a stent, preferably to both the interior and exterior surfaces of the implant. Preferably the polymer coating has a thickness ranging from 0.5 to 30 μm, more preferably 2 to 25 μm and most preferable 5 to 20 μm.

A pharmaceutical active loaded onto the implant has a dosage depending on its activity, its elution rate, and the desired elution period. The loading level depends on the claims of the polymer coating, affinity of the active for the polymer, the surface area of the coating and active loading method, and may be selected by the skilled person.

In a preferred embodiment, the implant is a flexible implant such as a stent; preferably comprising a generally tubular body having an interior and exterior wall. Preferably the stent is formed from a polymer material or a metal. Examples of suitable metals include but are not limited to tantalum, nitinol, cobalt chromium, cobalt-nickel-chromium, magnesium, titanium, and ferrous alloys such as stainless steel. The body may be formed of a laminate such as a steel-tantalum-steel laminate. Suitable polymer coating methods which leads to thicker external coatings are described in WO01/01957.

When developing a coating for an implant, there are various challenges which the skilled person has to overcome. More specifically, the coating has to be able to withstand various different processing conditions during its lifetime.

The first consideration is that it must be possible to apply the polymer to the implant in an even manner so as to form a complete homogenous film over the surface or surfaces as desired. Once applied to the surface and after the subsequent step (iv) of cross linking, the coating also needs to be robust enough to withstand general transport and handling. These properties are satisfied in the present invention.

In the case where the coated implant is a stent, the next stage in processing would be to attach the coated stent to a balloon catheter. Many different techniques can be used to attach the coated stent to achieve this. Often they involve bringing the two into intimate contact and securing them together by crimping or heat setting. The cross-linked polymer coating needs to be able to withstand the high temperatures and pressures involved in this. If the Tg of the polymer coating is too low, then the polymer may flow during the crimping process and form bridges between adjacent stent struts. The polymer coating according to the present invention avoids this by having a Tg which is very similar to or higher than the processing temperature at this stage. Furthermore, as discussed above, the incorporation of cross-links in the coating reduces the likelihood of flow and bridge-formation occurring.

Medical devices are usually subjected to a final stage of sterilisation prior to packaging. The polymer coating needs to be able to withstand the sterilisation process. It also needs to be stable upon storage. A problem encountered on storage is so called; “cold flow”, wherein the coating deforms over time as the polymer molecules undergo flow during storage. The polymer coating of the present invention avoids this by having a Tg when dry which is higher than storage conditions (25-30° C.) and which is cross-linked.

Prior to deployment in the body, the stent is tracked through the vasculature, which subjects the stent and the coating to abrasive forces. In addition, during deployment at the site of the lesion, the stent is expanded and the coating is subjected to significant expansion forces. For both steps, the coating needs to be flexible and tough. The coating provided in the present invention satisfies these requirements. The coated stent also needs to remain at the point of deployment and thus the coating should have long term chemical and dimensional stability in vivo. The coating provided in the present invention has such properties. Where the coating contains a pharmaceutical active, it should be mechanically both stable when the drug is present and after it has eluted. The polymer is also selected to have desirable equilibrium water content and rate of hydration. The polymer properties affect the drug loading and elution rates also. The present invention allows control of these properties.

In order to satisfy all of the above detailed considerations, preferably, once cross-linked, the biocompatible polymer has:
a) a measured Tg (dry) in the range from 15 to 90° C., preferably 15-70° C., preferably 20-50° C.;
b) a hydrated modulus at room temperature of more than 5 MPa, preferably more than 20 MPa
c) an elongation at break (hydrated) at 37° C. of more than 50%; preferably more than 150%, more preferably more than 250%; and
d) an equilibrium water content in the ranging from 10 to 60%.

As noted above, the present invention further provides an implant for permanent or temporary implantation into an animal body, e.g. into a body lumen. Preferably the implant is a stent having a tubular body having an interior wall and an exterior wall. Preferably the stent is metal. The surface of the implant is coated, preferably entirely coated, with a primer layer as defined above and a second biocompatible coating comprising a polymer as defined above which has been cross-linked. The biocompatible coating may further comprise pharmaceutical active(s) as defined above.

Where the coated implant is a coated stent, it may be subjected to further processing steps such as attachment onto a balloon catheter by, for example, crimping and heat setting. After this, the method may include a step of sterilisation. Sterilisation may be carried out by any known method including, for example, UV irradiation, gamma sterilisation, electron beam irradiation, autoclaving or ethylene oxide treatment. During a sterilisation step the polymer may be further cross-linked, i.e. to provide a second step which is part of the total cross-linking step (iv) of the process of the invention.

The present invention will now be described further with reference to the following figures and illustrative examples in which:

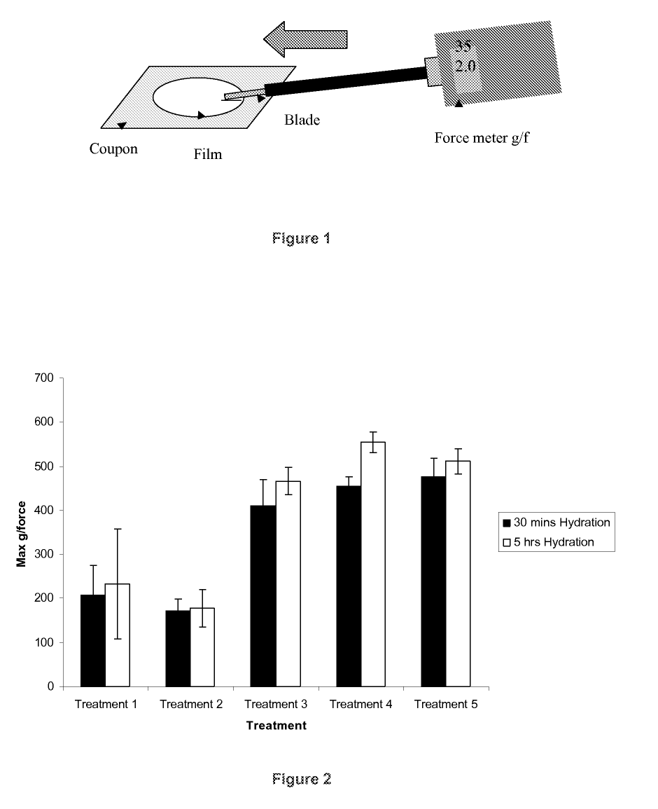

FIG. 1 is a schematic representation of the method of adhesion testing of the polymer;

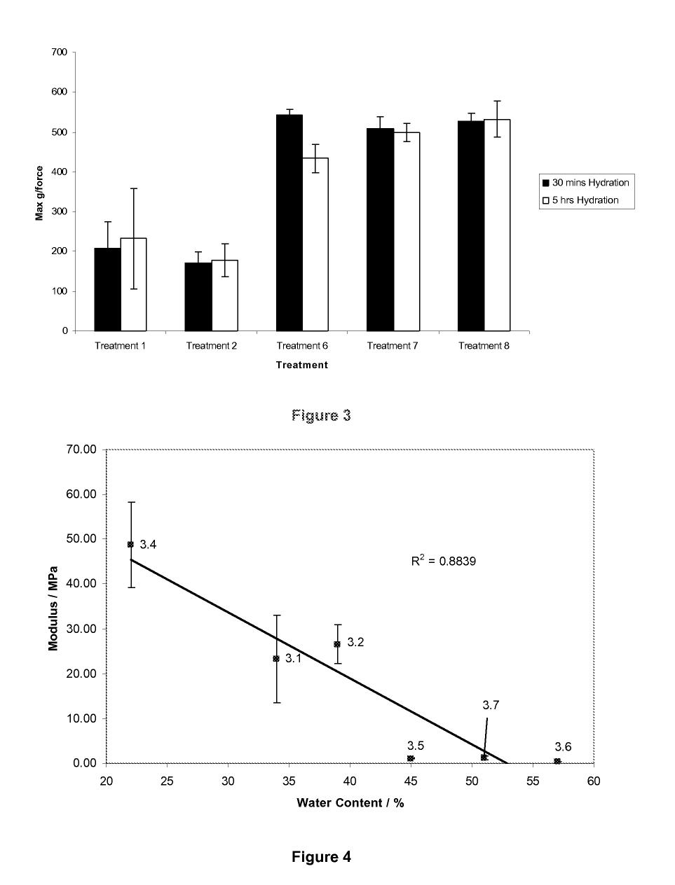

FIGS. 2 and 3 illustrate the results of adhesion testing as described in Example 6;

FIG. 4 illustrates the hydrated water content (room temperature) versus the modulus for a family of polymers;

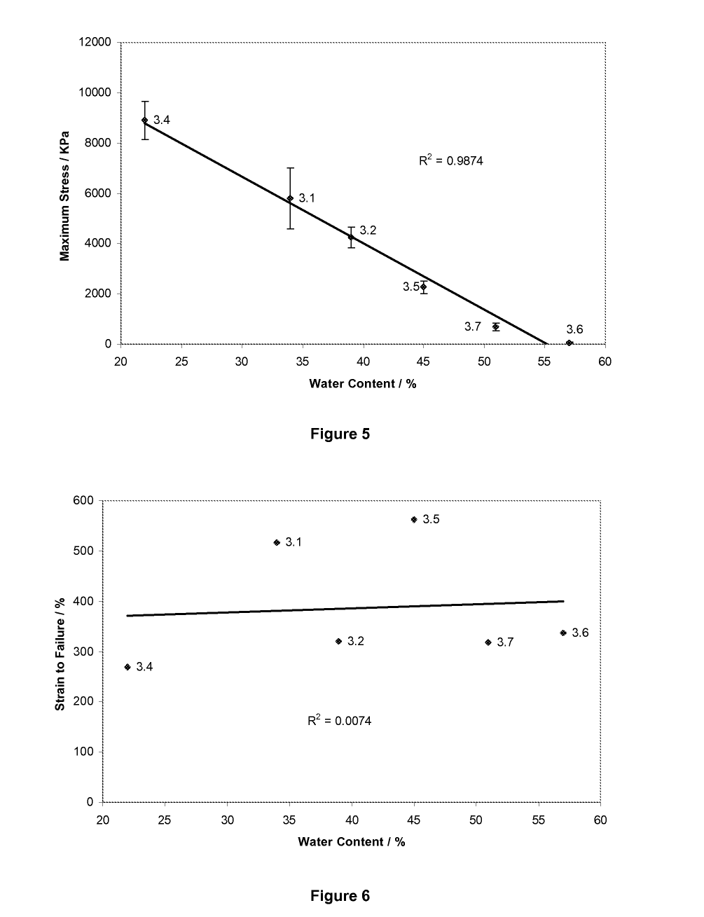

FIG. 5 illustrates the hydrated water content (room temperature) versus the maximum stress for a preferred family of polymers;

FIG. 6 shows the hydrated water content (room temperature) versus the strain to failure for a preferred family of polymers;

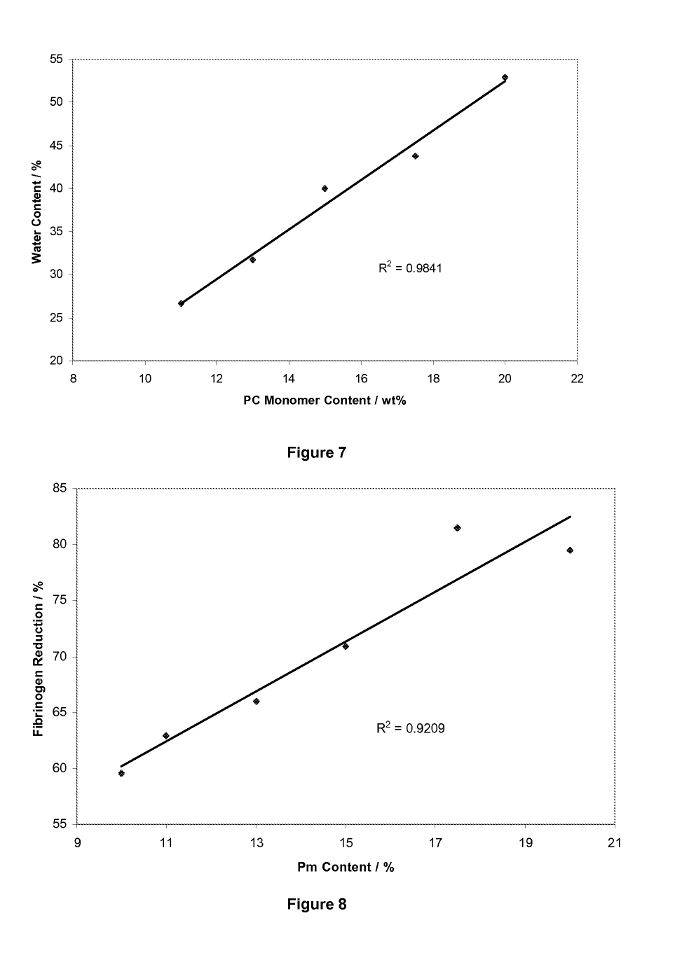

FIG. 7 shows the hydrated water content (room temperature) versus the content of monomer (i) for a preferred family of polymers;

FIG. 8 shows the fibrinogen binding reduction as a measure of the content of monomer (i) for a preferred family of polymers; and

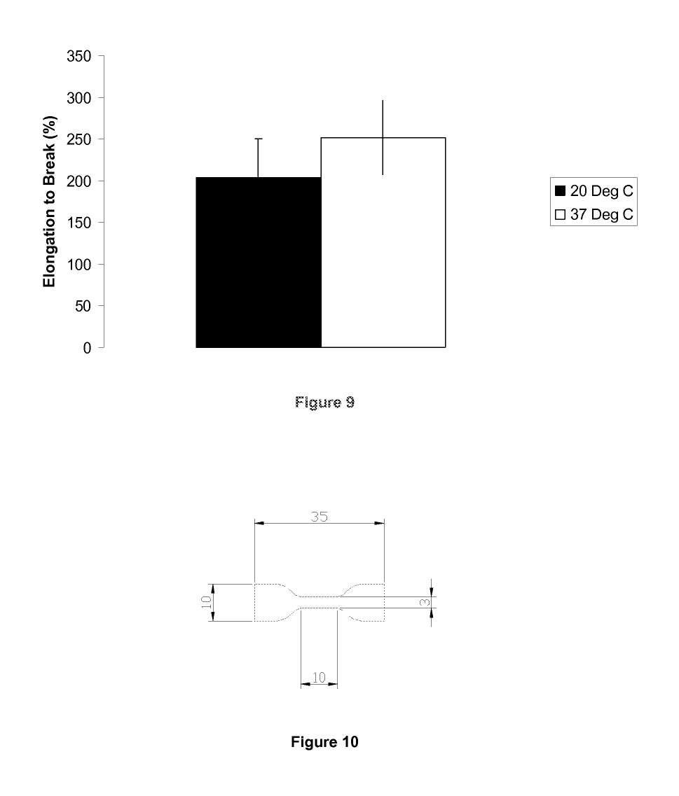

FIG. 9 shows the elongation at break for a polymer tested in the hydrated state at room temperature and 37° C.;

FIG. 10 shows the shape of a tensile test sample;

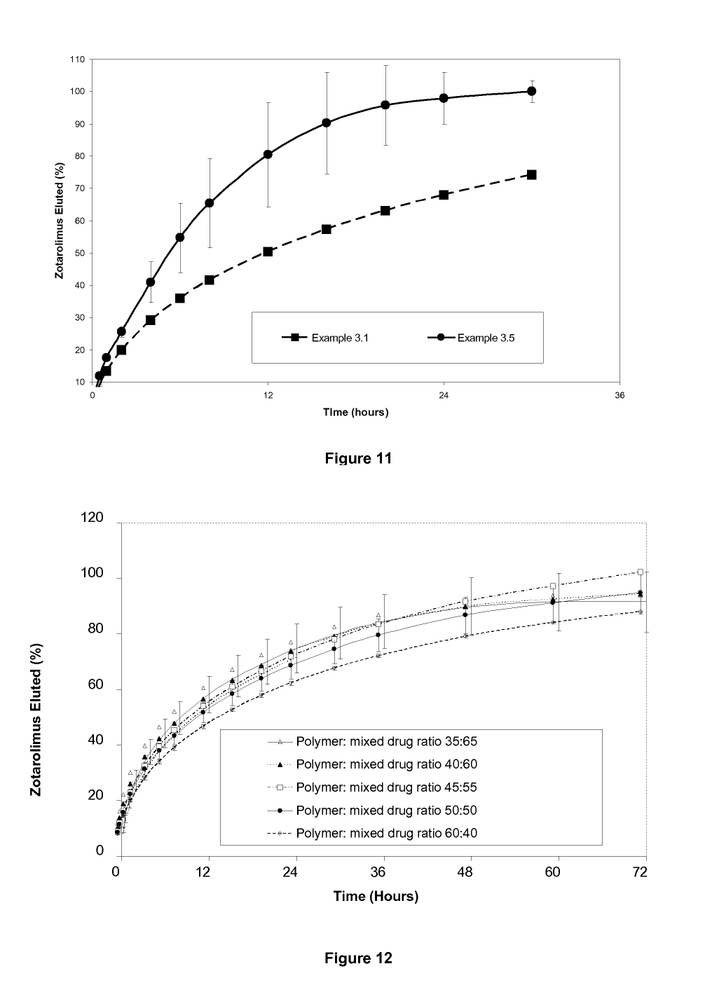

FIG. 11 shows the results of example 9; and

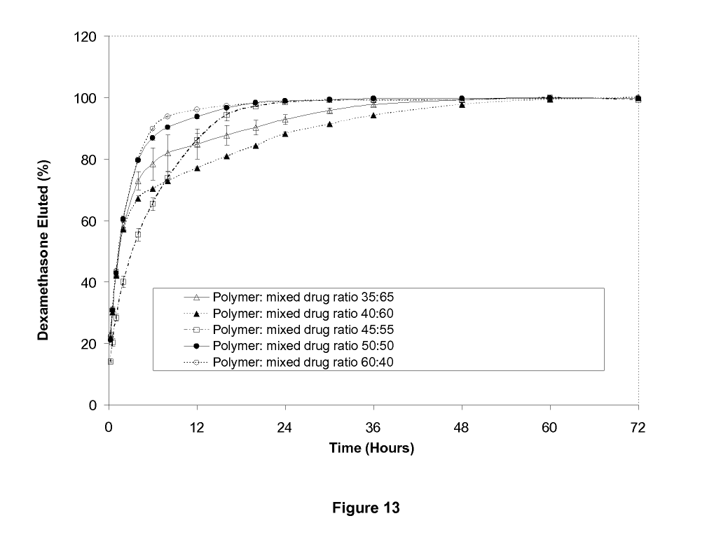

FIGS. 12 and 13 shows the results of example 10

### EXAMPLE 1

### Synthesis Of Polymers (Reference)

The polymer is made by free radical co-polymerisation using monomer feed conditions throughout the reaction. An azo compound is used during the reaction for initiation and the reaction mixture is heated under reflux as a solution in isopropanol.

The polymer is purified by precipitation and diafiltration. The diafiltration unit is a Millipore ProFlux unit with a 0.1 m² regenerated cellulose membrane having a molecular weight cut-off of 10 KDa.

Polymerisation and Precipitation

100 g of isopropanol was charged to a suitable multi-necked flask fitted with mechanical stirrer, thermometer, condenser and feed inlet. The temperature was raised to reflux (83° C.) using a heated oil bath. In a suitable stoppered flask a solution of 10 g (10 wt %) of PC monomer (2-methacryloyloxyethyl)-2′-(trimethylammonium)ethyl phosphate) in 10 g (100 wt %) of isopropanol was prepared. 15 g (15 wt %) of styrene, 15 g (15 wt %) methyl methacrylate, 40 g (40 wt %) butyl acrylate, 15 g (15 wt %) hydroxybutyl acrylate and 5 g (5 wt %) of trimethoxysilylpropyl methacrylate were then added.

A solution of 0.1 g of AIBN initiator in 3 g of isopropyl acetate was made. This initiator solution was added to the monomer solution using 3 g of isopropyl acetate as a rinse. The solution was well mixed and transferred to a 250 ml measuring cylinder using a further 5 g of isopropanol to rinse the flask.

When the reaction vessel solvent was refluxing the total monomer solution was pumped into the reaction flask over a period of 2 hours using a peristaltic pump.

The reaction mix was held at reflux for a further 75 minutes and then an initiator spike made up from 0.05 g of AIBN in 3 g of isopropyl acetate was added.

The reaction was held at reflux for a further 75 minutes and then a second spike made up from 0.05 g AIBN in 3 g isopropyl acetate was added.

The reaction was held at reflux for a further 150 minutes and then allowed to cool.

A 2 litre culture vessel fitted with mechanical stirrer and feed inlet was charged with the 500 ml of di-isopropyl ether. Using a suitable peristaltic pump, the polymer solution was pumped into this precipitation vessel with good but not vigorous stirring to avoid excessive splashing. As the precipitation proceeded, a further 1000 ml of di-isopropyl ether was added in four separate aliquots. When all the polymer solution had been added, the stirrer was turned off and the soft coagulated polymer mass allowed to settle. The supernatant solvents were decanted using a suction probe. 250 ml of di-isopropyl wash was added and stirred for 10 minutes. The stirrer was turned off and the supernatant solvent decanted. The wash was repeated with a further 250 ml of di-isopropyl ether and again as much of the supernatant as possible was decanted.

150 g of ethanol was added and after freeing the stirrer by hand the mixture was stirred until a clear solution was obtained. Gentle warming with an external warm water bath helped the dissolution. The polymer solution was transferred to a suitable tared bottle and a sample was taken for non-volatile content (110° C. for 30 minutes) to determine conversion.

Diafiltration

The Pellicon mini cassette and membrane was assembled and the whole ProFlux unit was flushed with about 300 ml of ethanol. The pump was started and the conditions adjusted to ensure that the membrane was well wetted and a significant amount of permeate was obtained. The system was drained. The polymer solution obtained was diluted with a further 150 g of ethanol and the solution was charged to the ProFlux reservoir. The pump was started and the conditions adjusted so that permeate was obtained at the rate of about 100 ml every 15-20 minutes. The diafiltration was continued until 200 ml of permeate had been collected. A further 1200 ml of ethanol was added to the ProFlux reservoir and the diafiltration was continued until 1200 ml of ethanol permeate had been collected.

The diafiltration was continued until an extra 150 ml of permeate had been collected and then the polymer solution was drained into a tared bottle. A sample of the final polymer solution was taken for non-volatile content and residual monomer and solvent composition analysis.

### EXAMPLE 2 (REFERENCE)

The general process of Example 1 was followed but using the formulation:

| | | |
|---|---|---|
| Isopropanol | 48 | g |
| Isopropyl acetate | 12 | g |
| PC monomer | 7.5 | g |
| Isopropanol | 48 | g |
| Methyl methacrylate | 7.5 | g |
| Butyl acrylate | 17.5 | g |
| Styrene | 7.5 | g |
| Hydroxypropyl methacrylate | 7.5 | g |
| Trimethoxypropylsilyl methacrylate | 2.5 | g |
| Azobismethylpropionitrile (AIBN) | 0.06 | g |
| Isopropyl acetate | 5.0 | g |
| Isopropyl acetate wash | 5.0 | g |
| AIBN | 0.02 | g |
| Isopropyl acetate | 5.0 | g |

The polymerisation was carried out as described in Example 1 and the resulting polymer solution precipitated in di-isopropyl ether (2 litres in all). The sticky precipitate was then dissolved in ethanol and the solids content adjusted to 20%. The solution was used without further purification by diafiltration. The mixture of mononers iii) has a calculated Tg of −7° C.

### EXAMPLE 3 (REFERENCE)

The process of Example 2 was followed with the following monomer compositions:

| MONOMER | Polymer | | | | | | |
|---|---|---|---|---|---|---|---|
| (Wt %) | 3.1 | 3.2 | 3.3 | 3.4 | 3.5 | 3.6 | 3.7 |
| PC Monomer | 10 | 10 | 15 | 10 | 15 | 15 | 29 |
| Styrene | 15 | 15 | 16 | 15 | 15 | — | — |
| Methyl | 20 | 20 | 16 | — | 15 | 15 | — |
| Methacrylate | | | | | | | |
| Lauryl | — | 35 | 33 | — | — | — | 51 |
| Methacrylate | | | | | | | |
| Butyl | — | — | — | 55 | — | 20 | — |
| Methacrylate | | | | | | | |
| Butyl Acrylate | 35 | — | — | — | 35 | 30 | — |
| Hydroxypropyl | — | — | 15 | — | — | — | 15 |
| Methacrylate | | | | | | | |
| Hydroxybutyl | 15 | 15 | — | 15 | 15 | 15 | — |
| Acrylate | | | | | | | |
| Trimeth- | 5 | 5 | 5 | 5 | 5 | 5 | 5 |
| oxysilylpropyl | | | | | | | |
| Methacrylate | | | | | | | |
| Tg of monomer | −23° C. | −30° C. | −11° C. | 4 | −33 | −17 | −44 |
| mix iii) | | | | | | | |

All polymers were precipitated in DIPE and then used without further purification. The polymer of approximately Example 3.1 has a measured Tg of 45° C.

### EXAMPLE 3.7

A polymer is formed of the phosphorylcholine monomer (29 parts by weight), lauryl methacrylate (51 parts), hydroxypropylmethacrylate (15 parts) and trimethoxylsilylpropyl methacrylate (5 parts) by the method described in detail in WO 9830615.

### EXAMPLE 4 (REFERENCE)

10 g of 1-butanol was placed in a suitable multi-necked flask fitted with mechanical stirrer, thermometer, condenser and feed inlet. The temperature was raised to reflux (118° C.) using a heated oilbath.

11 g of PC monomer was placed in a suitable stoppered flask and dissolved in 10 g of butanol.

20 g of styrene was added followed by 20 g of methyl methacrylate, 29 g of butyl acrylate, 14 g of hydroxybutyl acrylate and 6 g of trimethoxypropylsilyl methacrylate. The calculated Tg of the monomer mix iii) is −19° C.

2.0 g of a 5% solution of Luperox 331 M80 (Atofina Chemicals Inc.) initiator in butanol was also added and the solution mixed well.

The monomer solution was pumped to the refluxing butanol over a period of about 2 hours and then held at reflux for a further 75 minutes. An initiator spike of 1 g of the 5% solution of Luperox was then added and the reaction mix held at reflux for a further 2 hours.

After this time the polymer solution was cooled and diluted with 300 g of ethanol.

The diluted solution was then subjected to diafiltration through a regenerated cellulose membrane having a molecular weight cut off of 10K using a further 1500 ml of ethanol. The solution was finally concentrated to about 400 ml and decanted and the non-volatile content determined gravimetrically by evaporation of solvents at 100° C. The solution concentration was then adjusted to 29% by addition of a calculated amount of ethanol.

### EXAMPLE 5 (REFERENCE)

The polymer 3.7 is subjected to testing. The monomer mix iii) has a calculated Tg of −44° C., but the polymer contains no styrene. The modulus (hydrated) at room temperature is 1.25 MPa, while the modulus (hydrated) at 37° C. is 0.27 MPa. The equilibrium water content is 50° C. and the elongation at break is more than 200° C.

### EXAMPLE 6

### Adhesion to a Stainless Steel

Testing has shown that the presence of a silane primer improves the adhesion of the polymer of the type exemplified, specifically of Examples 3.1 and 3.7 to stainless steel surfaces. This testing involved two types of experiment. The first of these involved an assessment of the adhesion of the coating to stainless steel sheet. The second test involved placing strips of stainless steel sheet coated in the polymers of the type exemplified in Example 3.1 into saline or ethanol. The swelling behaviour of the coating was then evaluated over time. For each experiment two types of silane primer were used. The first was SP120 which is commercially available from Nusil Corporation, USA and the second silane used was based on bis[3-(trimethoxysilyl)-propyl]amine. The details of the sample preparation, test methods used and results are now described.

Sample Preparation

A sample of stainless steel (316L) was cut into small square sections. The surface of the stainless steel samples were then treated using either solvent cleaning, O₂ plasma treatment or both solvent cleaning and O₂ plasma treatment combined. Some samples were then coated in an adhesion promoter. The polymer of Example 3.1 was then added to the surface of each treated stainless steel sample and allowed to dry. Once dry, the polymer was cross-linked. For this experiment further details for each step is provided below.

Primer Solution Preparation: SP120

Mix 9.5 ml of ethanol to 0.5 ml of SP120 silane primer (Nusil Corporation, USA). Take 5 ml of this dilute SP120 solution and add to 45 ml of ethanol.

Primer Solution Preparation: Bis[3-(trimethoxysilyl)-propyl]amine

Mix 9 ml of ethanol to 0.5 ml of HPLC grade water. To this solution add 0.5 ml of Bis[3-(trimethoxysilyl)-propyl]amine. Take 5 ml of this dilute solution and add to 45 ml ethanol.

Surface Solvent Cleaning:

The surface solvent cleaning process used to clean the surface of the stainless steel strips involve a five-step cleaning process.

The steel coupons to be cleaned are first submerged in a beaker of fresh ultra-pure water (Romil). The beaker is then sonicated for 2 minutes. The water and coupons are then poured into a sieve and the liquid drained. The coupons are then placed into a clean beaker and absolute ethanol (Romil) added so that all the coupons are completely submerged. The beaker is then sonicated for 2 minutes. The ethanol and coupons are then poured into a sieve and the liquid drained. The coupons are then placed into a clean beaker and dichloromethane added so that all the coupons are completely submerged. The beaker is then sonicated for 2 minutes. The dichloromethane and coupons are then poured into a sieve and the liquid drained. The coupons were then placed into a clean beaker and dichloromethane (Romil) added so that all the coupons were completely submerged. The beaker is then sonicated for 2 minutes. The dichloromethane and coupons are then poured into a sieve and the liquid drained. The coupons are then placed into a clean beaker and ethanol absolute added so that all the coupons are completely submerged. The beaker is then sonicated for 2 minutes. The ethanol and coupons are then poured into a sieve and the liquid drained. The coupons are finally placed in a clean beaker and placed in an oven at 60-70° C. until completely dry

O₂ Plasma Treatment:

The coupons to be treated are placed on the shelf of the plasma etcher and then plasma treated for 300 seconds using O₂ plasma. The gas flow rate is set at 2-3 litres per minute and the plasma chamber is set to 200 watts.

The coupons are removed from the chamber using tweezers and used for the next stage within 30 minutes.

Application of the Primer:

Using a pipette, 50 μl of the primer solution is applied to one side of the coupon (making sure where applicable that this is the plasma treated side). Using tweezers, the coupon is manipulated to ensure full coverage with the primer. The primer is then allowed to dry at ambient temperature for approximately 5 minutes before the polymer is added

Polymer Coating

Using a pipette, 100 μl of polymer solution (200 mg/ml of polymer from Example 3.1 or polymer of example 3.7 in ethanol) was slowly applied to each coupon. The pipette was manipulated so that a square of approx 15 mm×15 mm of polymer was coated onto the centre of the coupon.

The polymer was then cross-linked for 4 hours at 70° C. in the presence of moisture. The presence of moisture during cross-linking is maintained by placing a beaker containing 400 ml of water in the oven with the samples.

Adhesion Testing

The samples were first allowed to hydrate in saline (0.9 Wt % NaCl) at room temperature for a minimum of 30 minutes prior to testing. Once hydrated the adhesion of the film to the stainless steel surface was tested. This was performed by measuring the force required to remove the coating from the steel surface. A force gauge with a modified 3 mm tip was used to measure the adhesion of the polymer to the stainless steel coupon. The coupon was placed onto a microscope stage. The tip of the stem of the force gauge was brought into contact with the edge of the polymer film at an angle of approximately 30°. The force gauge was set in compression mode and maximum peak mode and set to zero. The gauge was pushed forward until the film delaminated from the surface (FIG. 1). The peak force registered on the display was taken as the force required to remove the film from the stainless steel.

The test was repeated at two different hydration times (30 mins and 5 hours) and no significant differences were observed in the resulting adhesion test results between these two time points. Table C shows the different treatments studied and FIGS. 2 and 3 the results obtained for the SP120 silane and Bis[3-(trimethoxysilyl)-propyl]amine primer respectively. The polymer is from Example 3.1.

TABLE C
The different treatments applied to the stainless steel
coupons prior to coating with polymer from example 3.1.

| | Surface | | | |
|---|---|---|---|---|
| | Solvent | O₂ Plasma | SP120 | Bis [3-(trimethoxysilyl)- |
| Treatment | Cleaned | Treated | Primer | propyl]amine Primer |
| Treatment 1 | — | ✓ | — | — |
| Treatment 2 | ✓ | ✓ | — | — |
| Treatment 3 | — | ✓ | ✓ | — |
| Treatment 4 | ✓ | — | ✓ | — |
| Treatment 5 | ✓ | ✓ | ✓ | — |
| Treatment 6 | — | ✓ | — | ✓ |
| Treatment 7 | ✓ | — | — | ✓ |
| Treatment 8 | ✓ | ✓ | — | ✓ |

It can be seen from the data (FIGS. 2 and 3) that samples in which the stainless steel was simply cleaned as described generally gave lower adhesion values than for samples in which the surface had additionally been treated with the silane primer. This clearly shows that the presence of the primer increases the adhesion of the polymer coating to the surface of the stainless steel. In their hydrated state the polymer coatings were very tough and robust when handled and the steel sheets could be bent through 180° without the coating cracking or delaminating. Similar behaviours were also observed for the samples primed and coated with the polymer of Example 3.7 as the samples could also be bent with ease once hydrated through 180° without the coating showing signs of failure.

Forced Swelling Tests

The second test used to evaluate the adhesion of the polymer exemplified in Example 3.1 to stainless steel involved forced swelling of the coating. Prior to cross-linking, the polymer was readily soluble in ethanol. Therefore it was considered that this would be a suitable solvent to ‘force swell’ the coating from the stainless steel and if the coating could not be removed by swelling in ethanol, then it would be concluded that it had a strong adhesive bond to the stainless steel surface.

The different treatments applied to the stainless steel coupons prior to coating with the polymer from Example 3.1 are provided in Table N. Test samples of stainless steel strips coated in the polymer exemplified in Example 3.1 were prepared as described in the previous section.

As described earlier, once dry the coating was cross-linked at 70° C. for 4 hours in the presence of moisture. The samples were then immersed in ethanol and the appearance of the coating was evaluated over time. The data (see Table O) clearly show that the coating remains attached to the stainless steel surface in the form of an intact polymer film when the surface has been treated with a silane primer. Without a silane primer, the coating tended to swell from the surface and sometimes had a tendency to delaminate from the surface of the stainless steel.

TABLE N
The different treatments applied to the stainless steel
coupons prior to coating with polymer from Example 3.1.

| | Surface | | | |
|---|---|---|---|---|
| | Solvent | O₂ Plasma | SP120 | Bis [3-(trimethoxysilyl)- |
| Treatment | Cleaned | Treated | Primer | propyl]amine Primer |
| Treatment A | ✓ | — | — | — |
| Treatment B | ✓ | ✓ | — | — |
| Treatment C | ✓ | ✓ | ✓ | — |
| Treatment D | ✓ | ✓ | — | ✓ |

TABLE O
The results of swelling the polymer of Example 3.1 coatings
on stainless steel in ethanol at different time points.

| | Observations | Observations | Observations | Observations |
|---|---|---|---|---|
| | after 5 | after 15 | after 30 | after 60 |
| Treatment | minutes | minutes | minutes | minutes |
| Treatment A | Frosted surface | Coating very | Coating very | Coating very |
| | of coating | swollen but | swollen but | swollen but |
| | | still bonded | still bonded | still bonded |
| Treatment B | Frosted surface | Coating very | Coating very | Coating very |
| | of coating | swollen but | swollen but | swollen but |
| | | still bonded | still bonded | still bonded |
| Treatment C | Frosted surface | Good quality | Good quality | Good quality |
| | of coating | tightly bound | tightly bound | tightly bound |
| | | coating | coating | coating |
| Treatment D | Frosted surface | Good quality | Good quality | Good quality |
| | of coating | tightly bound | tightly bound | tightly bound |
| | | coating | coating | coating |

Aqueous Swelling Tests

A further test used to evaluate the adhesion of the polymers exemplified in Example 3.1 and the polymer of Example 3.7 to stainless steel involved the swelling of the coating in Isoton solution.

The different treatments applied to the stainless steel coupons prior to coating with the polymer from Examples 3.1 and 3.7 are provided in Table P. Test samples of stainless steel strips coated in the polymers exemplified in Examples 3.1 and 3.7 were prepared as described in the previous section.

As described earlier, once dry the coating was cross-linked at 70° C. for 4 hours in the presence of moisture. The samples were then immersed in Isoton solution and the appearance of the coating evaluated at different timepoints. At each time interval a sharp edge was used to try to prise the coating away from the surface of the stainless steel. A subjective assessment of the adhesion was made at each time point. The results are shown in Tables Q and U.

TABLE P
The different treatments applied to the stainless
steel coupons prior to coating with polymer.

| | Surface | | | |
|---|---|---|---|---|
| | Solvent | O₂ Plasma | SP120 | Bis [3-(trimethoxysilyl)- |
| Treatment | Cleaned | Treated | Primer | propyl]amine Primer |
| Treatment AA | ✓ | — | — | — |
| Treatment BB | ✓ | ✓ | — | — |
| Treatment CC | ✓ | ✓ | ✓ | — |
| Treatment DD | ✓ | ✓ | — | ✓ |
| Treatment EE | ✓ | ✓ | ✓ | — |
| Treatment FF | ✓ | ✓ | — | ✓ |

TABLE Q
Visual assessment of the adhesion of the Polymer from Example 3.1
to stainless steel coupons after incubation in Isoton over 24 hrs.

| | | Oxygen | | Incubation in |
|---|---|---|---|---|
| | Solvent | plasma | Silane | Isoton 25° C. |
| Treatment | cleaned | treatment | treatment | for 24 hrs |
| Treatment | ✓ | x | x | Good adhesion but |
| AA | | | | the film could be |
| | | | | removed using a |
| | | | | sharp edge |
| Treatment | ✓ | ✓ | x | Film fully |
| BB | | | | delaminated |
| Treatment | ✓ | x | SP120 | Well adhered film |
| CC | | | | |
| Treatment | ✓ | x | Bis[3(trimeth- | Well adhered film |
| DD | | | oxysilyl)- | |
| | | | propyl] amine | |
| Treatment | ✓ | ✓ | SP120 | Well adhered film |
| EE | | | | |
| Treatment | ✓ | ✓ | Bis[3(trimeth- | Well adhered film |
| FF | | | oxysilyl)- | |
| | | | propyl] amine | |

TABLE U
Visual assessment of the adhesion of the Polymer from Example 3.7
to stainless steel coupons after incubation in Isoton over 24 hrs.

| | | Oxygen | | Incubation in |
|---|---|---|---|---|
| | Solvent | plasma | Silane | Isoton 25° C. |
| Treatment | cleaned | treatment | treatment | for 24 hrs |
| Treatment | ✓ | x | x | Poor film adhesion. |
| AA | | | | Film swollen from |
| | | | | surface. |
| Treatment | ✓ | ✓ | x | Poor film adhesion. |
| BB | | | | Film swollen from |
| | | | | surface. |
| Treatment | ✓ | x | SP120 | Well adhered film |
| CC | | | | |
| Treatment | ✓ | x | Bis[3(trimeth- | Well adhered film |
| DD | | | oxysilyl)- | |
| | | | propyl] amine | |
| Treatment | ✓ | ✓ | SP120 | Well adhered film |
| EE | | | | |
| Treatment | ✓ | ✓ | Bis[3(trimeth- | Well adhered film |
| FF | | | oxysilyl)- | |
| | | | propyl] amine | |

The data from Tables Q and U clearly show that the coating remains attached to the stainless steel surface when placed in Isoton solution when the surface has been treated with a silane primer. In the case of the polymer exemplified in Example 3.1, it is difficult to remove the polymer film from the stainless steel with a sharp edge since the bonding is very strong. It was observed that for this polymer system, for Example 3.1, there was adhesion even when primers were not used and when the surface had only been solvent cleaned prior to coating with the polymer and cross-linking. The primer did however improve the adhesion of this polymer to the surface of stainless steel when compared to samples which had been only cleaned and/or plasma treated prior to coating with the polymer. Adhesion of the polymer of Example 3.7 was inadequate without primer, but good with both types of primer with or without plasma treatment.

### EXAMPLE 7

### Effect of Water Content of the Polymer (Reference)

The water content of the polymer films was measured according to the method detailed below.

The first step in the testing method is to cast a polymer film. PC polymer films were cast onto a sacrificial gelatine layer as it was not possible to remove the films from solid surfaces once dry.

The films were cast in 150 mm diameter glass petri dishes. The gelatine solution was prepared by dissolving 10 g of powdered gelatine in 100 ml of boiling water. Once dissolved, the gelatine solution was poured into the petri dishes. Enough solution was added to cover the surface of the dish. The dish was also tilted so that the gelatine coated the walls of the dish. The gelatine was then allowed to set and dry.

The polymer solution was prepared at a concentration of 200 mg/ml in ethanol. For a 150 mm diameter petri dish, 25 ml of solution was used; this produced a film approximately 0.35 mm thick. The polymer solution was poured onto the gelatine and allowed to dry at room temperature. Once dry, the films were cross-linked at 70° C. for 4 hours in the presence of moisture.

After cross-linking, the films were allowed to return to room temperature slowly, before being hydrated. This step was found to be important, as when the films were wetted while still being hot, the polymer film cracked. The films were hydrated using saline (0.9 wt % NaCl). After approximately 1 hour the gelatine had softened enough for the polymer film to be removed from the collective layer easily. The films were then kept in a hydrated state before testing. The same method as that used in Example 8 was used here to prepare and test the modulus and maximum stress.

The effect of the water content on the mechanical properties of polymer films at a given temperature has been studied. It can be seen that there are strong correlations between the water content and modulus, and between the water content and maximum stress (see FIGS. 4 and 5). The graphs show that both the Young's modulus (FIG. 4) and the maximum stress (FIG. 5) were inversely proportional to the water content. The graph showing strain to failure (FIG. 6) showed no relationship between water content and strain to failure.

These data show that the water content had a significant affect on the mechanical properties of the polymers, and therefore on the performance of the polymers on the stents. Therefore by changing the amount of hydrophilic high Tg monomer (i) incorporated into the polymer, the modulus and therefore the strength of the coating in its hydrated state can be controlled somewhat. The relationship between water content and the PC monomer content for a family of polymers is shown in FIG. 7.

The addition of further high Tg monomer (i), specifically in this case PC monomer, also increases the biocompatibility of the polymer film. By increasing the amount of PC monomer added it is possible to control the biocompatibility of the polymer coating. One way of assessing the biocompatibility of a coating is to measure the amount of fibrogen it takes up compared to control samples. A range of polymers were produced with the same monomers as the polymer which is exemplified in this Example 3.1, but with an increasing ratio of PC monomer. PC monomer was added to the monomer reaction mixtures from 10 to 20 wt % and the other monomers in the formulations were adjusted slightly in each case to account for the increased PC monomer. These polymers were tested for their equilibrium water content and their fibrinogen absorbance reduction (measured as in WO9301221) compared to PET sheet. These results are shown below in FIGS. 7 and 8 respectively.

It has surprisingly it has been found that good biocompatibility is observed even at relatively low amounts of monomer (i) PC monomer content despite the incorporation of significant levels of hydrophobic monomers (particularly styrene). The reductions involved are of a similar order to the reductions observed for the Example 3.7/5 polymer.

### EXAMPLE 8

### Flexibility (Reference)

As the stent is expanded in vivo, the stent coating needs to be designed to be flexible in the in vivo environment. Samples were therefore tested at both room temperature and at 37° C. (i.e. body temperature). The samples were tested according to the following methodology.

The first step in the testing method was to cast a polymer film. PC polymer films were cast onto a sacrificial gelatine layer as it was not possible to remove the films from solid surfaces once dry.

The films were cast in 150 mm diameter glass petri dishes. The gelatine solution was prepared by dissolving 10 g of powdered gelatine in 100 ml of boiling water. Once dissolved, the gelatine solution was poured into the petri dishes. Enough solution was added to cover the surface of the dish. The dish was also tilted so that the gelatine coated the walls of the dish. The gelatine was then allowed to set and dry.

The polymer solution was prepared at a concentration of 200 mg/ml in ethanol. For a 150 mm diameter petri dish, 25 ml of solution was used; this produced a film approximately 0.35 mm thick. The polymer solution was poured onto the gelatine and allowed to dry at room temperature. Once dry, the films were cross-linked at 70° C. for 4 hours in the presence of moisture.

After cross-linking, the films were allowed to return to room temperature slowly, before being hydrated. This step was found to be important, as when the films were wetted while still being hot, the polymer film cracked. The films were hydrated using saline (0.9 wt % NaCl). After approximately 1 hour the gelatine had softened enough for the polymer film to be removed from the collective layer easily. The films were then kept in a hydrated state before testing.

Tensile test coupons were then produced using a custom-made dumbbell cutter. The dimensions (in mm) of the coupon are shown below in FIG. 10. The test specimens were cut from the polymer film by firmly pressing the cutter into the hydrated film until a piece of the film was removed in the shape of a dumbell. The tests specimen were visually inspected to ensure that there were no surface imperfections. Samples with visible imperfections were not tested.

Tensile testing was performed using an Instron 4411 (or equivalent equipment). The following parameters were used during all the tests;

Flat faced pneumatic grips.

Grip separation—20 mm.

Load Cell—50N

Cross-head Speed—100 mm min−1

Since the modulus is sensitive to temperature, it is important that the temperature is controlled during testing. This can be accomplished by maintaining the saline used to hydrate the film during tensile testing at the desired temperature.

FIG. 9 shows the results obtained for the elongation to break of a sample of polymer from Example 3.1 at both room temperature and at 37° C. It can be seen that the elongation to break is increased as the temperature is raised. This is because the higher temperature is approaching the Tg of the polymer in its dry state and is effectively above the Tg of the hydrated and therefore plasticised polymer. This results in a significant increase in molecular flexibility of the polymer film or coating. This increase in molecular flexibility allows the polymer molecules to stretch to a greater extent under a given applied stress, and thus increases the strain to break.

### EXAMPLE 9

### Drug Elution from Coated Stents

Polymers as described in Examples 3.1 and 3.5 were combined with a pharmaceutical active, in this example the antiproliferative agent zotarolimus, in the drug to polymer ratio 40:60. These polymer drug combinations were applied using a spray coating technique (as in example 6) to oxygen plasma and silane-treated (as in example 6 using SP120) stainless steel stents. The coated stents were cured at 70° C. for 4 hrs followed by ethylene oxide sterilisation.

The coated stents were placed in 1% Solutol™ at 37° C. Aliquots of the elution media were taken at predetermined intervals between zero and 72 hrs. The concentration of pharmaceutical active in the aliquots of elution media were analysed using standard HPLC techniques. The levels of pharmaceutical active measured were plotted against the time points. FIG. II shows the cumulative total elution of zotarolimus in 1% Solutol at 37° C. from coronary stents coated with Example 3.1 and 3.5 in the pharmaceutical active to polymer ratio of 40:60.

### EXAMPLE 10

### Dual Elution of Drugs from Coated Stents

The polymer of Example 3.1 was combined with pharmaceutical actives, in this case zotarolimus and dexamethasone (50:50) weight ratio), in the total drugs to polymer weight ratios of 65:35, 60:40, 55:45, 50:50 and 40:60. These polymer-drug combinations were applied using a spray coating technique to oxygen plasma and silane-treated stainless steel stents of the techniques of example 6 and SP120 silane primer. The coated stents were cured at 70° C. for 4 hrs followed by ethylene oxide sterilisation.

The coated stents were placed in 1% Solutol™ at 37° C. Aliquots of the elution media were taken at predetermined intervals between zero and 72 hrs. The concentration of the pharmaceutical actives (zotarolimus and dexamethasone) in the aliquots of elution media were analysed using standard HPLC techniques. The levels of pharmaceutical active measured were plotted against the time points as shown in FIG. 12 which shows the coated stents were placed in 1% Solutol™ at 37° C. Aliquots of the elution media were taken at predetermined intervals between zero and 72 hrs. The concentration of the pharmaceutical actives (zotarolimus and dexamethasone) in the aliquots of elution media were analysed using standard HPLC techniques. The levels of pharmaceutical active measured were plotted against the time points as shown in FIG. 12 which shows The cumulative total elution of zotarolimus in 1% Solutol at 37° C. from coronary stents coated with Example 3.1 in the mixed drug to polymer ratios of 65:35, 60:40, 55:45, 50:50 and 40:60. FIG. 13 which shows the cumulative total elution of dexamethasone in 1% Solutol at 37° C. from coronary stents coated with Example 3.1 in the mixed drug to polymer ratios of 65:35, 60:40, 55:45, 50:50 and 40:60.

1. A method of forming a coated implant, where the implant has a surfaces comprising the following steps:
i) optionally cleaning the surface;
ii) optionally plasma treating the surface;
iii) coating the surface of the implant with a primer to form a primer layer;
iv) coating the primer layer formed in step iii) with a biocompatible polymer having a functional group capable of forming a covalent bond with the primer;
v) forming a covalent bond between the primer in the said primer layer and the biocompatible polymer by reacting said functional group of the biocompatible polymer with the primer; and
vi) covalently crosslinking the biocompatible polymer coating, wherein the primer includes a pendant group of general formula (II) 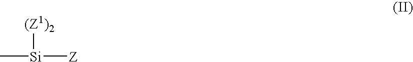
wherein Z is —OR³⁰ or Hal
Z¹ is —OR³⁰, Hal or C₁₋₁₂ alkyl
wherein R³⁰ is optionally hydroxy-substituted C₁₋ₓalkyl or acyl, x is 12 and Hal is a halogen atom.

2. The method according to claim 1, wherein the biocompatible polymer includes a pendant group of general formula (II) 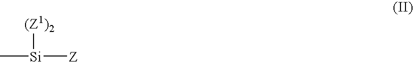
wherein Z is —OR³⁰ or Hal
Z¹ is —OR³⁰, Hal or C₁₋₁₂ alkyl
wherein R³⁰ is optionally hydroxy-substituted C₁₋ₓalkyl or acyl, x is 12 and Hal is a halogen atom.

3. The method according to claim 1, wherein the said pendant group of on the primer has general formula (IIA)
—Si(OR⁵)₃ (IIA)
wherein R⁵ is C₁₋₁₂ alkyl or C₂₋₁₂ acyl.

4. The method according to claim 1, wherein the primer comprises a mixture of a silicate, a titanate or zirconate and a silane having a pendant group of formula II.

5. The method according to claim 4, wherein the silane primer comprises a mixture of tetra-n-propyl silicate, tetrabutyl titanate and tetra (2-methoxyethoxy) silane in a solvent.

6. The method according to claim 3, wherein the primer comprise bis[3-(trimethoxysilyl)propyl]amine.

7. The method according to claim 1, wherein the primer layer is coated with the biocompatible polymer without any intermediate drying step.

8. The method according to claim 1, wherein the primer is dried prior to coating with the biocompatible polymer.

9. The method according to claim 1, wherein the primer is applied by a method selected from dip coating, spray coating and spin coating.

10. The method according to claim 2, wherein the biocompatible polymer is obtained by polymerising ethylenically unsaturated monomers including at least one monomer having the general formula (I) 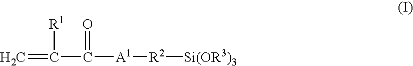
in which R¹ is hydrogen or C₁₋₄ alkyl;
A¹ is —O— or —NR⁴— wherein R⁴ is hydrogen or C₁₋₄ alkyl;
R² is C₁₋₂₄ straight or branched alkylene, alkylene oxaalkylene or alkylene oligoxaalkylene in which the alkylene group has 1 to 6 carbon atoms; and
each R³ is independently selected from C₁₋₆ alkyl groups.

11. The method according to claim 10, wherein the ethylenically unsaturated monomers include a zwitterionic monomer.

12. The method according to claim 11, wherein the zwitterionic monomer is of the general formula (III):
YBX (III)
wherein
B is a straight or branched alkylene (alkanediyl), alkyleneoxaalkylene or alkylene oligooxaalkylene chain optionally containing one or more fluorine atoms up to and including perfluorinated chains or, if X or Y contains a terminal carbon atom bonded to B, a valence bond;
X is a zwitterionic group; and
Y is an ethylenically unsaturated polymerisable group selected from 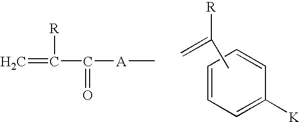
CH₂═C(R)CH₂O—, CH₂═C(R)CH₂OC(O)—, CH₂═C(R)OC(O)—, CH₂═C(R)O—, CH₂═C(R)CH₂OC(O)N(R⁶)—, R⁷OOCCR═CRC(O)O—, RCH═CHC(O)O—, RCH═C(COOR⁷)CH₂C(O)O—, 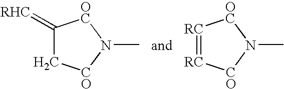
wherein:
R is hydrogen or a C₁-C₄ alkyl group;
R⁶ is hydrogen or a C₁-C₄ alkyl group or R⁷ is —B—X where B and X are as defined above; and
R⁷ is hydrogen or a C₁₋₄ alkyl group;
A is —O— or —NR⁶—;
K is a group —(CH₂)ₚOC(O)—, —(CH₂)ₚC(O)O—,
—(CH₂)ₚOC(O)O—, —(CH₂)ₚNR⁸—, —(CH₂)ₚNR⁸C(O)—,
—(CH₂)ₚC(O)NR⁸—, —(CH₂)ₚNR⁸C(O)O—, —(CH₂)ₚOC(O)NR⁸—,
—(CH₂)ₚNR⁸C(O)NR⁸— (in which the groups R⁸ are the same or different), —(CH₂)ₚO—, —(CH₂)ₚSO₃—, or, optionally in combination with B, a valence bond
p is from 1 to 12; and
R⁸ is hydrogen or a C₁-C₄ alkyl group.

13. The method according to claim 12, wherein X is a group of formula (VIII) 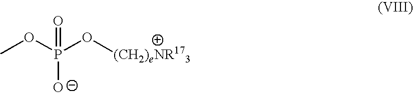
wherein the groups R¹⁷ are the same or different and each is hydrogen or C₁₋₄ alkyl and e is from 1 to 4.

14. The method according to claim 13, wherein each group R¹⁷ is methyl and e is 2.

15. The method according to claim 11, wherein the ethylenically unsaturated monomers include comonomer selected from C₁₋₂₄ alkyl(alk)acrylates and -(alk)acrylamides and analogues having hydroxyl or (oligo) alkoxy substituents on the C₁₋₂₄ alkyl groups.

16. The method according to claim 1, wherein the biocompatible polymer coating is applied by dipping the implant into a solution of the polymer in a solvent and evaporating the solvent.

17. The method according to claim 1, wherein the polymer coating is applied by spraying the surface(s) of the implant with a solution of the polymer in a solvent.

18. The method according to claim 16, wherein the polymer solution further comprises a pharmaceutical active.

19. The method according to claim 1 wherein the implant is generally tubular and both inner and outer surfaces of the implant are coated with the polymer.

20. The method according to claim 1, wherein in (vi), the polymer is crosslinked by application of heat and/or moisture.

21. The method according to claim 1, wherein the surface of the implant has been is plasma treated.

22. The method according to claim 21, wherein the surface of the implant has been is plasma treated with an oxygen plasma.

23. The method according to claim 1, which includes a step (i) of cleaning the implant.

24. The method according to claim 1, wherein the implant is a stent comprising a generally tubular body formed of an impermeable material having an interior wall and an exterior wall.

25. An implant for permanent or temporary implantation into a body lumen, having a surface coated with
(i) a first coating of a primer material formed from a compound including a pendant group of general formula (II) 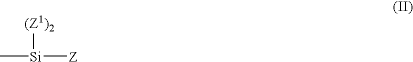
wherein Z is —OR³⁰ or Hal
Z¹ is —OR³⁰, Hal or alkyl
wherein R³⁰ is optionally hydroxy substituted C₁₋₁₂ alkyl or acyl and Hal is a halogen atom; and
(ii) a second biocompatible coating over the primer coating comprising a cross-linked polymer covalently bonded to the primer material.

26. The implant according to claim 25, wherein the biocompatible polymer is formed from a compound including a pendant group of general formula (II) 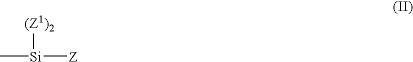
wherein Z is —OR³⁰ or Hal
Z¹ is —OR³⁰, Hal or alkyl
wherein R³⁰ is optionally hydroxy substituted C₁₋₁₂ alkyl or acyl and Hal is a halogen atom.

27. The implant according to claim 25, wherein the said pendant group of the primer material has general formula (IIA)
—Si(OR⁵)₃ (IIA)
wherein R⁵ is C₁₋₁₂ alkyl or acyl.

28. The implant according to claim 26, wherein the biocompatible polymer the has pendant groups of formula IIA
—Si(OR⁵)₃ (IIA)
wherein R⁵ is C₁₋₁₂ alkyl or acyl.

29. The implant according to claim 27, wherein the primer is formed from a mixture of tetra-n-propyl silicate; tetrabutyl titanate and tetra (2-methoxyethoxyl) silane in a solvent.

30. The implant according to claim 27, wherein the primer comprises bis[3-(trimethoxysilyl)propyl]amine.

31. The implant according to claim 28, wherein the crosslinked polymer has been obtained by copolymerizing ethylenically unsaturated monomers including at least one monomer having the general formula (I) 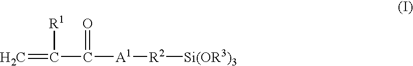
in which R¹ is hydrogen or C₁₋₄ alkyl;
A¹ is —O— or —NR⁴— wherein R⁴ is hydrogen or C₁₋₄ alkyl;
R² is C₁₋₂₄ straight or branched alkylene, alkylene oxaalkylene or alkylene oligoxaalkylene in which the alkylene group has 1 to 6 carbon atoms; and
each R³ is independently selected from C₁₋₆ alkyl groups.

32. The implant according to claim 31, wherein the ethylenically unsaturated monomers include zwitterionic monomer.

33. The implant according to claim 32, wherein the zwitterionic monomer is of the general formula (III)
YBX (III)
wherein
B is a straight or branched alkylene (alkanediyl), alkyleneoxaalkylene or alkylene oligooxaalkylene chain optionally containing one or more fluorine atoms up to and including perfluorinated chains or, if X or Y contains a terminal carbon atom bonded to B, a valence bond;
X is a zwitterionic group; and
Y is an ethylenically unsaturated polymerisable group selected from 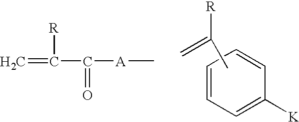
CH₂═C(R)CH₂O—, CH₂═C(R)CH₂OC(O)—, CH₂═C(R)OC(O)—, CH₂═C(R)O—, CH₂═C(R)CH₂OC(O)N(R⁶)—, R⁷OOCCR═CRC(O)O—, RCH═CHC(O)O—,
RCH═C(COOR⁷)CH₂C(O)O—, 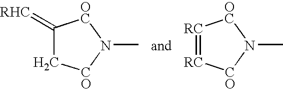
wherein:
R is hydrogen or a C₁-C₄ alkyl group;
R⁶ is hydrogen or a C₁-C₄ alkyl group or R⁶ is —B—X where B and X are as defined above; and
R⁷ is hydrogen or a C₁₋₄ alkyl group;
A is —O— or —NR⁶—;
K is a group —(CH₂)ₚOC(O)—, —(CH₂)ₚC(O)O—,
—(CH₂)ₚOC(O)O—, —(CH₂)ₚNR⁸—, —(CH₂)ₚNR⁸C(O)—,
—(CH₂)ₚC(O)NR⁸—, —(CH₂)ₚNR⁸C(O)O—, —(CH₂)ₚOC(O)NR³—,
—(CH₂)ₚNR⁸C(O)NR⁸— (in which the groups R⁸ are the same or different), —(CH₂)ₚO—, —(CH₂)ₚSO₃—, or, optionally in combination with B, a valence bond
p is from 1 to 12; and
R³ is hydrogen or a C₁-C₄ alkyl group.

34. The implant according to claim 33, wherein X is a group of formula
(VIII) 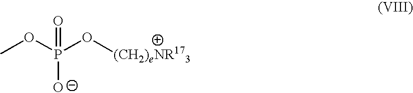
where the groups R¹⁷ are the same in different and each is hydrogen or C₁₋₄ alkyl and e is from 1 to 4.

35. The implant according to claim 34, wherein each group R¹⁷ is methyl and e is 2.

36. The implant according to claim 31, wherein the ethylenically unsaturated monomers include comonomer selected from C₁₋₂₄ alkyl(alk)acrylates and -(alk)acrylamides and analogues having hydroxyl or (oligo) alkoxy substituents on the C₁₋₂₄ alkyl groups.

37. (canceled)

38. The implant according to claim 25, which is a stent comprising a generally tubular body formed of metal having an interior wall and an exterior wall.

39. The stent according to claim 38, wherein the metal is selected from stainless steel, nitinol and tantalum.

40. The implant according to claim 25, wherein the biocompatible coating further comprises pharmaceutical active(s).

41. The implant according to claim 25, wherein the thickness of the biocompatible coating is in the range of from 0.5 to 50 μm.

42. The method according to claim 12, in which Y is 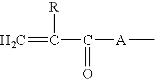
wherein R is methyl and A is O.

43. The method of claim 11, wherein the ethylenically unsaturated monomers include comonomer selected from hydroxy C₁₋₂₄ (meth)acrylates.

44. The method of claim 2, wherein the said pendant group on the biocompatible polymer has general formula IIA
—Si(OR⁵)₃ (IIA)
wherein R⁵ is C₁₋₁₂ alkyl or C₂₋₁₂ acyl.

45. The method of claim 14, wherein the ethylenically unsaturated monomers include comonomer selected from hydroxy C₁₋₂₄ (meth)acrylates.

46. The implant according to claim 33, in which Y is 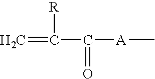
wherein R is methyl and A is O.

47. The implant according to claim 36, wherein the ethylenically unsaturated monomers include comonomer selected from hydroxy C₁₋₂₄ (meth)acrylates.

48. The implant according to claim 35, wherein the ethylenically unsaturated monomers include comonomer selected from hydroxy C₁₋₂₄ (meth)acrylates.

49. The method according to claim 17, wherein the polymer solution further comprises a pharmaceutical active.
